(19) United States
(12) Patent Application Publication
(10) Pub. No.: US 2024/0084040 A1
(43) Pub. Date: Mar. 14, 2024

(54) NOVEL CARBOHYDRATE ANTIBODIES, PHARMACEUTICAL COMPOSITIONS AND USES THEREOF

(71) Applicant: OBI PHARMA, INC., TAIPEI CITY (TW)

(72) Inventors: Jiann-Shiun LAI, TAIPEI CITY (TW); Hui-Wen CHANG, TAIPEI CITY (TW); Yin-Chieh KUO, TAIPEI CITY (TW); Chi-Sheng HSIA, TAIPEI CITY (TW); Woan Eng CHAN, TAIPEI CITY (TW); Ming-Tain LAI, TAIPEI CITY (TW)

(21) Appl. No.: 18/264,563

(22) PCT Filed: Feb. 9, 2022
(86) PCT No.: PCT/US2022/015834

§ 371 Date: Nov. 17, 2023

Related U.S. Application Data
(60) Provisional application No. 63/147,441, filed on Feb. 9, 2021.

(51) Int. Cl. C07K 16/46; A61P 35/00

(52) U.S. Cl. CPC C07K 16/468; A61P 35/00; C07K 2317/24; C07K 2317/31; C07K 2317/565

(57) ABSTRACT

The present invention provides antibody or the antigen-binding portion thereof bind to carbohydrate antigen, such as Globo series antigens (e.g. Globo H, SSEA-4 or SSEA-3). Also disclosed herein are pharmaceutical compositions and methods for the inhibition of cancer cells in a subject in need thereof. The pharmaceutical compositions comprise an antibody or an antigen-binding portion thereof and at least one pharmaceutically acceptable carrier.

### CROSS-REFERENCE TO RELATED APPLICATION

This application claims the benefit of priority from U.S. Provisional Application No. 63/147,441, filed Feb. 9, 2021, the entire content of which is incorporated herein by reference.

### FIELD

The present invention relates to modified antibodies to carbohydrate antigens, having specific amino acid substitutions relative to the unmodified antibodies. The present invention also relates to the use of these antibodies in the treatment, prevention or management of diseases or disorders, such as cancer or the inhibition of cancer cells.

### BACKGROUND

Numerous surface carbohydrates are expressed in malignant tumor cells. For example, Globo H (Fuc α1→2Galβ1→3GalNAcβ1→3Gal α1→4Galβ1→4Glc) has been shown to be overexpressed on a variety of epithelial cancers and is associated with tumor aggressiveness and poor prognosis in breast cancer and small cell lung carcinoma. Previous studies have shown that Globo H and stage-specific embryonic antigen 3 (Galβ1→3GalNAcβ1→3Galα1→4Galβ1→4Glcβ1) (SSEA-3, also called Gb5) were observed on breast cancer cells and breast cancer stem cells (Chang W W et al., (2008) PNAS, 105 (33):11667-11672; Cheung S K et al., (2016) PNAS, 113 (4):960-965). In addition, SSEA-4 (stage-specific embryonic antigen-4) (Neu5Acα2→3Galβ1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al., (1983) EMBO J, 2:2355-2361). These findings support that Globo series antigens (Globo H, SSEA-3, and SSEA-4) are unique targets for cancer therapies and can be used to direct therapeutic agents in targeting cancer cells effectively.

Some earliest antibodies were mouse monoclonal antibodies (mAbs), secreted by hybridomas prepared from lymphocytes of mice immunized with these Globo series antigens. However, there are problems associated with the use of mouse antibodies in human, such as inability to trigger certain human effector function and adverse reaction including cytokine releases syndrome. Antibodies derived from a nonhuman species are humanized to enhance the effector function and/or lower the adverse reaction. However, a humanized antibody with most optimal binding activity and pharmacokinetic value, such as in vivo T_{1/2} or half life, has not been reported.

There is still an unmet need to optimize the pharmacokinetic value of a humanized antibody. The present invention provides antibodies with optimized pharmacokinetic value to satisfy these and other needs.

### SUMMARY OF THE INVENTION

In certain embodiment, the mouse monoclonal anti-Globo H antibody (designated as the “2C2” antibody) and humanized anti-Globo H antibody (designated as the “OBI-888” antibody) are as described in PCT patent publications (WO2015157629A2 and WO2017062792A1), the contents of which are incorporated by reference in its entirety.

The present invention provides for antibodies, or antigen-binding portions thereof, comprising a variable region that bind to Globo series antigens (Globo H, stage-specific embryonic antigen 3 (SSEA-3), and stage-specific embryonic antigen 4 (SSEA-4).

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 1, 2 and 3 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 4, 5 and 6 respectively. Furthermore, the antibody comprises (a) a heavy chain variable region, having amino acid sequence about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 19 and (b) a light chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 20, wherein the antibody is designated as the “R783” antibody.

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 7, 8 and 9 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 10, 11 and 12 respectively. Furthermore, the antibody comprises (a) a heavy chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 21 and (b) a light chain variable region, having amino acid sequence about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 22, wherein the antibody is designated as the “R725-2” antibody.

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 13, 14 and 15 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 16, 17 and 18 respectively. Furthermore, the antibody comprises (a) a heavy chain variable region, having amino acid sequence about 90%, about 91%, about 92%, about 93%, about 94%, about 95%, about 96%, about 97%, about 98%, about 99% or about 100% identical to the amino acid sequence set forth in SEQ ID NO: 23 and (b) a light chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 24, wherein the antibody is designated as the “R643” antibody.

Some embodiments provide pharmaceutical compositions comprising the antibody or antigen-binding portion thereof as described herein and at least one pharmaceutically acceptable carrier.

Some embodiments also provide for methods of inhibiting cancer cells, comprising administering to a subject in need thereof an effective amount of the antibody or antigen-binding portion thereof described herein. In one embodiment, the cancer cells are Globo H expressing cancer cells which include, but are not limited to, sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer and prostate cancer.

### BRIEF DESCRIPTION OF THE DRAWINGS

FIG. 1 illustrates the amino acid sequences of six anti-Globo H antibodies designated as 2C2, OBI-888, 82V, R783, R725-2 and R643. Panel A illustrates the amino acid sequences of the heavy chain variable regions and the CDRs of the anti-Globo H antibodies and panel B illustrates the amino acid sequences of the light chain variable regions and the CDRs of the anti-Globo H antibodies.

FIG. 2 is a line graph illustrating the pharmacokinetic profile of the OBI-888, 82V and R783 antibodies in mouse.

FIG. 3 is line graph illustrating the pharmacokinetic profile of the OBI-888 and R783 antibodies in monkey.

FIG. 4 illustrates the result of an efficacy study of the OBI-888, R783 and R725-2 antibodies in the MCF-7 xenograft model.

FIG. 5 illustrates the cytotoxicity of the OBI-888, 82V, R783 and R725-2 antibodies using CD56+ NK cell (Panel A: 12.5 nM, Panel B: 50 nM; Panel C: 200 nM).

FIG. 6 illustrates the phagocytosis of the OBI-888 and R783 antibodies. Antibody pre-treated target cells and healthy donor macrophage were co-cultured in a serum free medium for 1 hour (Panel A) and 3 hours (Panel B).

### DETAILED DESCRIPTION OF THE INVENTION

As used herein, the articles “a” and “an” refer to one or more than one (i.e., at least one) of the grammatical object of the article. By way of example, “an element” means one element or more than one element.

An “effective amount,” as used herein, refers to a dose of the antibody or pharmaceutical composition that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

The term “subject” can refer to a vertebrate having cancer or to a vertebrate deemed to be in need of cancer treatment. Subjects include all warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

The term “antibody” is intended to encompass antibodies, digestion fragments, specified portions and variants thereof, including antibody mimetics or comprising portions of antibodies that mimic the structure and/or function of an antibody or a specified fragment or portion thereof, including single chain antibodies and fragments thereof, each containing at least one CDR derived from an antibody of the present invention. Antibodies include antibody fragments, antibody variants, monoclonal antibodies, polyclonal antibodies, and recombinant antibodies and the like. Antibodies can be generated in mice, rabbits or humans.

The antibodies can be full-length or can comprise a fragment (or fragments) of the antibody having an antigen-binding portion, including, but not limited to, Fab (e.g., by papain digestion), Fab′ (e.g., by pepsin digestion and partial reduction) and F(ab′)₂ (e.g., by pepsin digestion), Facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques), bivalent scFv (bi-scFv), trivalent scFv (tri-scFv), Fd, dAb fragment (e.g., Ward et al., Nature, 341:544-546 (1989)), an isolated CDR, diabodies, triabodies, tetrabodies, linear antibodies, single-chain antibody molecules, bispecific and multispecific antibodies formed from antibody fragments.

Single chain antibodies produced by joining antibody fragments using recombinant methods, or a synthetic linker, are also encompassed by the present invention. Bird et al., Science, 1988, 242:423-426. Huston et al., Proc. Natl. Acad. Sci. USA, 1988, 85:5879-5883.

Multispecific or bi-specific antibodies or fragments thereof may be specific for different epitopes of one target carbohydrate (e.g., Globo H) or may contain antigen-binding domains specific for more than one target carbohydrate (e.g., antigen-binding domains specific for Globo H, SSEA-3 and SSEA-4). In one embodiment, a multispecific antibody or antigen-binding portion thereof comprises at least two different variable domains, wherein each variable domain is capable of specifically binding to a separate carbohydrate antigen or to a different epitope on the same carbohydrate antigen. Tutt et al., 1991, J. Immunol. 147:60-69. Kufer et al., 2004, Trends Biotechnol. 22:238-244. The antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. In one embodiment, the bi-specific antibody comprises a first binding domain that binds to a Globo series antigen and a second binding domain that specifically binds to a T cell surface antigen. In some embodiments, the tumor associated carbohydrate antigen is a Globo series antigen. In some embodiments, the T cell surface antigen is CD2, CD3, CD4, CD5, CD6, CD8, CD28, CD40L or CD44.

The antibodies or antigen-binding portions may be peptides. Such peptides can include variants, analogs, orthologs, homologs and derivatives of peptides, that exhibit a biological activity, e.g., binding to a tumor-associate carbohydrate antigen. The peptides may contain one or more analogs of an amino acid (including, for example, non-naturally occurring amino acids, amino acids which only occur naturally in an unrelated biological system, modified amino acids from mammalian systems etc.), peptides with substituted linkages, as well as other modifications known in the art.

The antibody, or antigen-binding portion thereof, can be derivatized or linked to another functional molecule. For example, an antibody can be functionally linked (by chemical coupling, genetic fusion, noncovalent interaction, etc.) to one or more other molecular entities, such as another antibody, a detectable agent, a cytotoxic agent, a pharmaceutical agent, a protein or peptide that can mediate association with another molecule (such as a streptavidin core region or a polyhistidine tag), amino acid linkers, signal sequences, immunogenic carriers, or ligands useful in protein purification, such as glutathione-S-transferase, histidine tag, and staphylococcal protein A. One type of derivatized protein is produced by crosslinking two or more proteins (of the same type or of different types). Suitable cross linkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill. Useful detectable agents with which a protein can be derivatized (or labeled) include fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

An antibody light or heavy chain variable region comprises a framework region (FW) interrupted by three hypervariable regions, referred to as complementarity determining regions or CDRs. According to one aspect of the invention, the antibody or the antigen-binding portion thereof may have the following structure:
Leader Sequence-FW1-CDR1-FW2-CDR2-FW3-CDR3-
wherein the amino acid sequences of CDR1, CDR2 and CDR3 of the present invention are disclosed in Table 1.

The heavy chain and light chain variable regions of the present antibodies or antigen-binding portions thereof can be from a non-human or human source. The framework of the present antibodies or antigen-binding portions thereof can be human, humanized, non-human (e.g., a murine framework modified to decrease antigenicity in humans), or a synthetic framework (e.g., a consensus sequence).

Antibodies of the present invention also include chimerized or humanized monoclonal antibodies, generated from non-human (e.g., murine) antibodies of a hybridoma. Also encompassed by the present invention are antibodies or antigen-binding portions thereof comprising one or two variable regions as disclosed herein, wherein certain sequences of the variable region, such as the framework sequence, replaced by sequences from at least one different species including, but not limited to, human, rabbits, sheep, dogs, cats, cows, horses, goats, pigs, monkeys, apes, gorillas, chimpanzees, ducks, geese, chickens, amphibians, reptiles and other animals.

The term “humanized antibody” refers to an antibody comprising at least one human framework and at least one, preferably all CDRs from a non-human antibody, and in which any constant region present is substantially identical to a human antibody constant region, i.e., about 85-90%, at least about 90%, at least about 95% identical. Hence, all parts of a humanized antibody, except possibly the CDR, are substantially identical to corresponding parts of one or more human antibody sequences. Humanized antibodies can be generated by replacing sequences of the variable region that are not directly involved in antigen binding (e.g., framework) with equivalent sequences from human variable regions. Techniques for obtaining humanized antibodies are routinely available to the skilled person, they have been described, inter alia, in U.S. Pat. Nos. 5,225,539; 6,548,640; and 6,982,321. Those techniques are well known in the art, include isolating, manipulating, and expressing the nucleic acid sequences that encode all or part of variable regions from at least one of a heavy or light chain. For example, once non-human (e.g., murine) antibodies are obtained, variable regions can be sequenced, and the location of the CDRs and frameworks residues determined. Kabat, E. A., et al. (1991) Sequences of Proteins of Immunological Interest, Fifth Edition, U.S. Department of Health and Human Services, NIH Publication NO. 91-3242. Chothia, C. et al. (1987) J. Mol. Biol., 196:901-917. DNA encoding the light and heavy chain variable regions can, optionally, be ligated to corresponding constant regions and then subcloned into an appropriate expression vector. CDR-grafted antibody molecules can be produced by CDR-grafting or CDR substitution. One, two, or all CDRs of an immunoglobulin chain can be replaced. For example, all of the CDRs of a particular antibody may be from at least a portion of a non-human animal or only some of the CDRs may be replaced. It is only necessary to keep the CDRs required for binding of the antibody to a predetermined carbohydrate antigen (e.g., Globo H). Morrison, S. L., 1985, Science, 229:1202-1207. Oi et al., 1986, BioTechniques, 4:214. U.S. Pat. Nos. 5,585,089; 5,225,539; 5,693,761 and 5,693,762. EP 519596. Jones et al., 1986, Nature, 321:552-525. Verhoeyan et al., 1988, Science, 239:1534. Beidler et al., 1988, J. Immunol., 141:4053-4060.

A chimeric antibody is a molecule in which different portions are derived from different animal species. For example, a chimeric antibody may contain a variable region derived from a murine mAb and a human immunoglobulin constant region. Human constant region DNA sequences can be isolated in accordance with well-known procedures from a variety of human cells (see Kabat et al., 1991; and WO 87/02671). Chimeric antibodies can be produced by recombinant DNA techniques. Morrison, et al., Proc Natl Acad Sci, 81:6851-6855 (1984). For example, a gene encoding a murine (or other species) antibody molecule is digested with restriction enzymes to remove the region encoding the murine Fc, and the equivalent portion of a gene encoding a human Fc constant region is then substituted into the recombinant DNA molecule. Chimeric antibodies can also be created by recombinant DNA techniques where DNA encoding murine V regions can be ligated to DNA encoding the human constant regions. Better et al., Science, 1988, 240:1041-1043. Liu et al. PNAS, 1987 84:3439-3443. Liu et al., J. Immunol., 1987, 139:3521-3526. Sun et al. PNAS, 1987, 84:214-218. Nishimura et al., Canc. Res., 1987, 47:999-1005. Wood et al. Nature, 1985, 314:446-449. Shaw et al., J. Natl. Cancer Inst., 1988, 80:1553-1559. International Patent Publication NOs: WO1987002671 and WO 86/01533. European Patent Application NOs: 184, 187; 171,496; 125,023; and 173,494. U.S. Pat. No. 4,816,567.

All antibody isotypes are encompassed by the present invention, including IgG (e.g., IgG1, IgG2, IgG3, IgG4), IgM, IgA (IgA1, IgA2), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

The terms “wild type antibody” and “unmodified antibody” are used interchangeably and as used herein refer to an antibody comprising an amino acid sequence which lacks one or more of amino acid substitutions disclosed herein.

In another exemplary embodiment, antibodies may have amino acid substitutions in the CDRs, such as to improve binding affinity of the antibody to the antigen. In yet another exemplary embodiment, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in Bowie et al., Science, 247:1306-1310 (1990). Cunningham et al., Science, 244:1081-1085 (1989). Ausubel (ed.), Current Protocols in Molecular Biology, John Wiley and Sons, Inc. (1994). T. Maniatis, E. F. Fritsch and J. Sambrook, Molecular Cloning: A Laboratory Manual, Cold Spring Harbor laboratory, Cold Spring Harbor, N.Y. (1989). Pearson, Methods Mol. Biol. 243:307-31 (1994). Gonnet et al., Science 256:1443-45 (1992).

According to one aspect of the invention, the amino acid substitutions described herein occur at positions corresponding to the Kabat numbering scheme (e.g., Kabat et al., Sequences of Immunological Interest. 5th Ed. Public Health Service, National Institutes of Health, Bethesda, Md. (1991)).

Antibodies, or antigen-binding fragments, variants or derivatives thereof of the present disclosure can also be described or specified in terms of their binding affinity to an antigen. “Affinity” refers to the strength of the sum total of noncovalent interactions between a single binding site of a molecule (e.g., an antibody) and its binding partner (e.g., a tumor associated carbohydrate). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd) Affinity can be measured by common methods known in the art, including those described herein. The affinity of an antibody for a carbohydrate antigen can be determined experimentally using any suitable method, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); or ELISA method.

The present antibodies or antigen-binding portions thereof can be produced by host cells transformed with DNA encoding light and heavy chains (or portions thereof) of a desired antibody. Antibodies can be isolated and purified from these culture supernatants and/or cells using standard techniques. For example, a host cell may be transformed with DNA encoding the light chain, the heavy chain, or both, of an antibody. Recombinant DNA technology may also be used to remove some or all of the DNA encoding either or both of the light and heavy chains that is not necessary for binding, e.g., the constant region. DNA can be expressed in various suitable cells, including prokaryotic and eukaryotic cells, e.g., bacterial cells, (e.g., E. coli), yeast cells, plant cells, insect cells, and mammalian cells. A number of mammalian cell lines are known in the art and include immortalized cell lines available from the American Type Culture Collection (ATCC). Non-limiting examples of the cells include all cell lines of mammalian origin or mammalian-like characteristics, including but not limited to, unmodified cells, derivatives and/or engineered variants of monkey kidney cells (COS, e.g., COS-1, COS-7), HEK293, baby hamster kidney (BHK, e.g., BHK21), Chinese hamster ovary (CHO), NS0, PerC6, BSC-1, human hepatocellular carcinoma cells (e.g., Hep G2), SP2/0, HeLa, Madin-Darby bovine kidney (MDBK), myeloma and lymphoma cells. The engineered variants include, e.g., glycan profile modified and/or site-specific integration site derivatives.

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 1, 2 and 3 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 4, 5 and 6 respectively. Furthermore, the antibody comprises (a) a heavy chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 19 and (b) a light chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 20, wherein the antibody is designated as the “R783” antibody.

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 7, 8 and 9 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 10, 11 and 12 respectively. Furthermore, the antibody comprises (a) a heavy chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 21 and (b) a light chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 22, wherein the antibody is designated as the “R725-2” antibody.

In one embodiment, the present invention provides for an antibody, or an antigen-binding portion thereof, that binds to a carbohydrate antigen or a fragment thereof and comprises a heavy chain variable region, wherein the heavy chain variable region comprises three complementarity determining regions (CDRs), HCDR1, HCDR2 and HCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 13, 14 and 15 respectively. This invention also provides for an antibody, or an antigen-binding portion thereof, that binds to a carbohydrate antigen or a fragment thereof and comprises a light chain variable region, wherein the light chain variable region comprises three CDRs, LCDR1, LCDR2 and LCDR3, having amino acid sequences about 90%, about 91%, about 92%, about 93%, about 94%, about 95%, about 96%, about 97%, about 98%, about 99% or about 100% identical to the amino acid sequence set forth in SEQ ID NOs: 16, 17 and 18 respectively. Furthermore, the antibody comprises (a) a heavy chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 23 and (b) a light chain variable region, having amino acid sequences about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the amino acid sequence set forth in SEQ ID NO: 24, wherein the antibody is designed as the “R643” antibody.

All numbers herein are approximations and may be modified by “about”, which is defined as ±1%.

In some embodiments, the humanized anti-Globo H antibody (OBI-888) or the antigen-binding portions thereof is a non-human antibody obtained from the hybridoma designated as 2C2 (deposited under ATCC Accession No.: PTA-121138). See WO2015157629A2, the content of which is incorporated by reference in its entirety.

Table 1 shows the amino acid sequences of the heavy chain variable region, the light chain variable region, and the CDRs of the modified antibodies (R783 antibody, R725-2 antibody and R643 antibody).

TABLE 1
Sequences of anti-Globo H antibody (R783, R725-2 and R643)

| | | | SEQ ID |
|---|---|---|---|
| Antibody | Chain Region | Amino Acid Sequence | NO. |
| R783 | first heavy chain | GFSLY RFDMGVG | 1 |
| | complementarity | | |
| | determining region | | |
| | (HCDR1) | | |
| R783 | second heavy chain | HIWWDDDKYYNPALKSR | 2 |
| | complementarity | | |
| | determining region | | |
| | (HCDR2) | | |
| R783 | third heavy chain | VRGLHDYYYYF | 3 |
| | complementarity | | |
| | determining region | | |
| | (HCDR3) | | |
| R783 | first light chain | CQASEDVSYM | 4 |
| | complementarity | | |
| | determining region | | |
| | (LCDR1) | | |
| R783 | second light chain | YGTSNKA | 5 |
| | complementarity | | |
| | determining region | | |
| | (LCDR2) | | |
| R783 | third light chain | CQQWSRRP | 6 |
| | complementarity | | |
| | determining region | | |
| | (LCDR3) | | |
| R725-2 | HCDR1 | GFSLY RFDMGVG | 7 |
| R725-2 | HCDR2 | HIWWDDDKYYNPALQSR | 8 |
| R725-2 | HCDR3 | VRGLHDYYYYF | 9 |
| R725-2 | LCDR1 | CQASEDVSYM | 10 |
| R725-2 | LCDR2 | YGTSNKA | 11 |
| R725-2 | LCDR3 | CQQWSRRP | 12 |
| R643 | HCDR1 | GFSLY RFDMGVG | 13 |
| R643 | HCDR2 | HIWWDDDKYYNPALQSR | 14 |
| R643 | HCDR3 | VRGLHDYYYYF | 15 |
| R643 | LCDR1 | CQASEDVSYM | 16 |
| R643 | LCDR2 | YGTSNKA | 17 |
| R643 | LCDR3 | CQQWSRRP | 18 |
| R783 | heavy chain variable | QITLQESGPTLVKPTQTLTLTCTFSGFSLYRFDM | 19 |
| | domain (V_{H}) | GVGWIRQPPGQGLEWLAHIWWDDDKYYNPAL | |
| | | KSRLTISKDTSKNQVVLTMTNMDPVDTATYYC | |
| | | ARVRGLHDYYYYFAYWGQGTLVTVSS | |
| R783 | light chain variable | EIVLTQSPSTLSLSPGERATLSCQASEDVSYMH | 20 |
| | domain (V_{L}) | WYQQKPGQAPQPWIYGTSNKASGVPSRFSGSG | |
| | | SGTDFTLTISSLQPEDVATYYCQQWSRRPFTFG | |
| | | QGTKVEIKR | |
| R725-2 | heavy chain variable | QITLQESGPTLVKPTETLTLTCAFSGFSLYRFDM | 21 |
| | domain (V_{H}) | GVGWIRQPPGQGLEWLAHIWWDDDKYYNPAL | |
| | | QSRLTISKDTSKNQVVLTMTNMDAEDTATYYC | |
| | | ARVRGLHDYYYYFAYWGQGTLVTVSS | |
| R725-2 | light chain variable | EIVLTQSPSTLSLSPGERATLSCQASEDVSYMH | 22 |
| | domain (V_{L}) | WYQQKPGQAPQPWIYGTSNKASGVPSRFSGSG | |
| | | SGTDFTLTISSLQPEDVATYYCQQWSRRPFTFG | |
| | | QGTKVEIKR | |
| R643 | heavy chain variable | QITLQESGPTLVKPTETLTLTCAFSGFSLYRFDM | 23 |
| | domain (V_{H}) | GVGWIRQPPGQGLEWLAHIWWDDDKYYNPAL | |
| | | QSRLTISKDTSKNQVVLTMTNMDAEDTATYYC | |
| | | ARVRGLHDYYYYFAYWGQGTLVTVSS | |
| R643 | light chain variable | EIVLTQSPSTLSLSPGESVTLSCQASEDVSYMH | 24 |
| | domain (V_{L}) | WYQQKPGQAPQPWIYGTSNKASGVPSRFSGSG | |
| | | SGTDFTLTISSLEPEDVATYYCQQWSRRPFTFG | |
| | | QGTKVEIER | |

In one embodiment, the modified antibody is generated by substituting one or more amino acid residues of a CDR of a wild type/unmodified 2C2 antibody. Such modified antibody may be conveniently generated using phage display-based affinity maturation techniques. Briefly, several hypervariable region sites (e.g. 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g. binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such modified antibodies are generated, the panel of variants is subjected to screening using techniques known in the art, including those described herein, and modified antibodies with superior properties in one or more relevant assays may be selected for further development.

The modified antibodies may also be produced by methods described, for example, by Marks et al., 1992, (affinity maturation by variable heavy chain (VH) and variable light chain (VL) domain shuffling), or Barbas, et al., 1994; Shier et al., 1995; Yelton et al., 1995; Jackson et al., 1995; and Hawkins et al., 1992 (random mutagenesis of CDR and/or framework residues).

The modified antibodies or antigen-binding portions thereof have in vitro and in vivo therapeutic, prophylactic, and/or diagnostic utilities. For example, these modified antibodies can be administered to cells in culture, e.g., in vitro or ex vivo, or to a subject, e.g., in vivo, to treat, inhibit, prevent relapse, and/or diagnose diseases, such as cancer.

Antibodies or the antigen binding portions thereof of the present invention are capable of modulating, decreasing, antagonizing, mitigating, alleviating, blocking, inhibiting, abrogating and/or interfering with at least one tumor-associate carbohydrate antigen or a fragment thereof in vitro, in situ and/or in vivo.

The invention also provides methods for inhibiting the growth of a cell in vitro, ex vivo or in vivo, wherein the cell, such as a cancer cell, is contacted with an effective amount of an antibody or an antigen-binding portion thereof as described herein. Pathological cells or tissue such as hyperproliferative cells or tissue may be treated by contacting the cells or tissue with an effective amount of an antibody or an antigen-binding portion thereof of this invention. The cells, such as cancer cells, can be primary cancer cells or can be cultured cells available from tissue banks such as the American Type Culture Collection (ATCC). The pathological cells can be cells of a Globo H expressing cancer, gliomas, meningioma, pituitary adenomas, or a CNS metastasis from a systemic cancer, lung cancer, prostate cancer, breast cancer, hematopoietic cancer or ovarian cancer. The cells can be from a vertebrate, preferably a mammal, more preferably a human. U.S. Patent Publication No. 2004/0087651. Balassiano et al. (2002) Intern. J. Mol. Med. 10:785-788. Thorne, et al. (2004) Neuroscience 127:481-496. Fernandes, et al. (2005) Oncology Reports 13:943-947. Da Fonseca, et al. (2008) Surgical Neurology 70:259267. Da Fonseca, et al. (2008) Arch. Immunol. Ther. Exp. 56:267-276. Hashizume, et al. (2008) Neuroncology 10:112-120. In one embodiment, the cancer is Globo H expressing cancer. In another embodiment, the cancer is SSEA-3 expressing cancer. In yet another embodiment, the cancer is SSEA-4 expressing cancer. Globo H expressing cancer, SSEA-3 expressing cancer and SSEA-4 expressing cancer include one or more of sarcoma, skin cancer, leukemia, lymphoma, brain cancer, glioblastoma, lung cancer, breast cancer, oral cancer, head-and-neck cancer, nasopharyngeal cancer, esophageal cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer and/or prostate cancer. In one aspect, the method comprises the assaying of a sample selected from one or more of breast, ovary, lung, pancreatic, stomach (gastric), colorectal, prostate, liver, cervix, esophagus, brain, oral, and/or kidney cancer.

In vitro efficacy of the present antibody or the antigen-binding portion thereof can be determined using methods well known in the art. MTT assay is based on the principle of uptake of MTT, a tetrazolium salt, by metabolically active cells where it is metabolized into a blue colored formazan product, which can be read spectrometrically. J. of Immunological Methods 65:55 63, 1983. The cytotoxicity of the antibody or the antigen-binding portion thereof may be studied by colony formation assay. Functional assays for binding Globo H antigen may be performed via ELISA. Cell cycle block by the antibody or the antigen-binding thereof may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays. Assays can also be done in vivo using a murine model. See, e.g., B. Teicher, Tumor Models for Efficacy Determination. Mol Cancer Ther 2006; 5:2435-2443.”

The present invention also provides pharmaceutical compositions comprising an antibody or antigen-binding portion thereof described herein, and a pharmaceutically acceptable carrier. Pharmaceutically acceptable carriers include any and all solvents, dispersion media, isotonic and absorption delaying agents, and the like that are physiologically compatible. In one embodiment, the pharmaceutical composition is effective to inhibit cancer cells in a subject.

Routes of administration of the present pharmaceutical compositions include, but are not limited to, intravenous, intramuscular, intranasal, subcutaneous, oral, topical, subcutaneous, intradermal, transdermal, subdermal, parenteral, rectal, spinal, or epidermal administration.

The pharmaceutical compositions of the present invention can be prepared as injectables, either as liquid solutions or suspensions, or as solid forms which are suitable for solution or suspension in liquid vehicles prior to injection. The pharmaceutical composition can also be prepared in solid form, emulsified or the active ingredient encapsulated in liposome vehicles or other particulate carriers used for sustained delivery. For example, the pharmaceutical composition can be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the pharmaceutical composition.

Naturally, the pharmaceutical compositions to be used for in vivo administration must be sterile; sterilization may be accomplished be conventional techniques, e.g. by filtration through sterile filtration membranes. It may be useful to increase the concentration of the antibody to come to a so-called high concentration liquid formulation (HCLF); various ways to generate such HCLFs have been described.

The pharmaceutical composition is administered alone, and/or mixed with another therapeutic agent, for example, a second monoclonal or polyclonal antibody or the antigen-binding portion thereof, a cancer vaccine or an anti-cancer agent such as DNA damaging or tubulin binding agents, or agents which inhibit angiogenesis, signal transduction pathways or mitotic checkpoints. The combination product may be a mixture of the two ingredients, or they may be covalently attached. In one example, the antibody or antigen-binding portion thereof specifically binds to Globo H is combined with an antibody (monoclonal or polyclonal) or antigen-binding portion thereof specifically binds VEGF. In another example, the second agent is a chemotherapy agent (e.g., cyclophosphamide, 5-fluorouracil or Actinomycin-D). The antibodies can also be administered in combinations with a cancer vaccine, e.g., Globo H conjugated with Diphtheria Toxin and a saponin adjuvant. The additional therapeutic agent may be administered simultaneously with, optionally as a component of the same pharmaceutical preparation, or before or after administration of the claimed antibody of the invention. Actual methods of preparing such dosage forms are known, or will be modified, to those skilled in the art. See, e.g., Remington's Pharmaceutical Sciences, Mack Publishing Company, Easton, Pennsylvania, 21st edition.

Pharmaceutical compositions can be administered in a single dose treatment or in multiple dose treatments on a schedule and over a time period appropriate to the age, weight and condition of the subject, the particular composition used, and the route of administration, whether the pharmaceutical composition is used for prophylactic or curative purposes, etc. For example, in one embodiment, the pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an antibody according to the invention, e.g., the period of time over which the pharmaceutical composition is administered, can vary, depending on any of a variety of factors, e.g., subject response, etc. For example, the pharmaceutical composition can be administered over a period of time ranging from about one or more seconds to one or more hours, one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

For ease of administration and uniformity of dosage, oral or parenteral pharmaceutical compositions in dosage unit form may be used. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The data obtained from the cell culture assays and animal studies can be used in formulating a range of dosage for use in humans. In one embodiment, the dosage of such compounds lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Sonderstrup, Springer, Sem. Immunopathol. 25:35-45, 2003. Nikula et al., Inhal. Toxicol. 4 (12):123-53, 2000.

An exemplary, non-limiting range for a therapeutically or prophylactically effective amount of an antibody or antigen-binding portion of the invention is from about 0.001 to about 60 mg/kg body weight, about 0.01 to about 30 mg/kg body weight, about 0.01 to about 25 mg/kg body weight, about 0.5 to about 25 mg/kg body weight, about 0.1 to about 20 mg/kg body weight, about 10 to about 20 mg/kg body weight, about 0.75 to about 10 mg/kg body weight, about 1 to about 10 mg/kg body weight, about 2 to about 9 mg/kg body weight, about 1 to about 2 mg/kg body weight, about 3 to about 8 mg/kg body weight, about 4 to about 7 mg/kg body weight, about 5 to about 6 mg/kg body weight, about 8 to about 13 mg/kg body weight, about 8.3 to about 12.5 mg/kg body weight, about 4 to about 6 mg/kg body weight, about 4.2 to about 6.3 mg/kg body weight, about 1.6 to about 2.5 mg/kg body weight, about 2 to about 3 mg/kg body weight, or about 10 mg/kg body weight.

The pharmaceutical composition is formulated to contain an effective amount of the present antibody or antigen-binding portion thereof, wherein the amount depends on the animal to be treated and the condition to be treated. In one embodiment, the present antibody or antigen-binding portion thereof is administered at a dose ranging from about 0.01 mg to about 10 g, from about 0.1 mg to about 9 g, from about 1 mg to about 8 g, from about 2 mg to about 7 g, from about 3 mg to about 6 g, from about 10 mg to about 5 g, from about 20 mg to about 1 g, from about 50 mg to about 800 mg, from about 100 mg to about 500 mg, from about 0.01 μg to about 10 g, from about 0.05 μg to about 1.5 mg, from about 10 μg to about 1 mg protein, from about 30 μg to about 500 μg, from about 40 μg to about 300 μg, from about 0.1 μg to about 200 μg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy and can be determined by one of ordinary skill in the art without undue experimentation.

The present antibodies, antigen-binding portions thereof, pharmaceutical compositions and methods of use are applicable and can be used in all vertebrates, e.g., mammals and non-mammals, including human, mice, rats, guinea pigs, hamsters, dogs, cats, cows, horses, goats, sheep, pigs, monkeys, apes, gorillas, chimpanzees, rabbits, ducks, geese, chickens, amphibians, reptiles and other animals.

The following examples of specific aspects for carrying out the present invention are offered for illustrative purposes only and are not intended to limit the scope of the present invention in any way.

### EXAMPLES

### Example 1. Generation of Modified Antibody

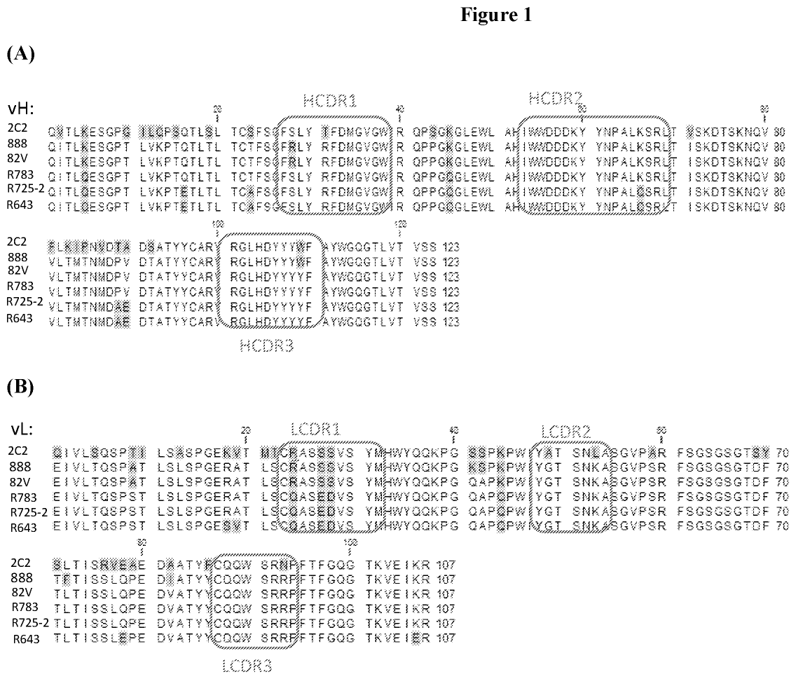

To enhance the stability/half-life of original anti-Globo H antibody (2C2 and OBI-888), the pI value (Isoelectric point) of the original anti-Globo H antibody was lowered through sequence mutation of heavy chain variable domain (V_{H}) and light chain variable domain (V_{L}). FIG. 1 illustrated sequence comparison between six anti-Globo H antibodies (2C2, OBI-888, 82V, R783, R725-2 and R643). Four antibodies 82V, R783, R725-2 and R643 were generated from single point mutation through sequence simulation in order to lower the pI value. There was only one amino acid variation in the HCDR1 (sequence identity 91.7%) and one amino acid variation in the HCDR3 (sequence identity 90.9%) between the OBI-888 antibody and R783 antibody (Panel A). Furthermore, there were three amino acid variations in the LCDR1 (sequence identity 70%) between the OBI-888 antibody and R783 antibody (Panel B). The pI values of these antibodies were calculated as follows:
pI value of OBI-888=8.7; pI value of 2C2=8.57; pI value of 82V=8.64; pI value of R783=7.83; pI value of R725-2=7.25; pI value of R643=6.43.

### Example 2. Binding Affinity of Anti-Globo H Antibody with SPR (Surface Plasmon Resonance) Analysis

### Experimental Material

Chip: Series S Sensor Chip SA (Cytiva, Cat. NO. BR100531)
Ligand: Globo H-PEG12-Biotin
Analyte: Anti-Globo H antibody
HBS-EP⁺ Buffer 10× (Cytiva, Cat. NO. BR100669)
Regeneration buffer: 10 mM Glycine, pH 2.0 (Cytiva, Cat. NO. BR100355)
Analyzed by Biacore T200 (Cytiva)

### Experimental Method

### (1) Buffer Preparation

1. Precondition buffer (total 1200 mL): 20 mL of 3M NaOH+240 mL of 5M NaCl+940 mL dd H₂O.
2. Coating solution (200 pg/mL Globo H-PEG12-Biotin): using 2 mg/mL stock solution to dilute as 200 pg/mL with dd H₂O.
3. Running buffer: Diluting 10× HBS-EP⁺ Buffer to 1× and filtering with 0.22 mm filter.

### (2) Immobilization

1. Step 1: Pumping precondition buffer into SPR chip channel path 1, 2 within 1 minute by the flow rate 30 mL/min and waiting for 1 minute.
2. Repeating step 1 for five times.
3. Step 2: Pumping coating solution into SPR chip channel path 2 within 3 minutes by the flow rate 30 mL/min and waiting for 1 minute.
3. Step 3: Pumping Glycine solution (pH 2.0) into SPR chip channel path 1, 2 within 1 minute by the flow rate 30 mL/min and waiting for 1 minute.

### (3) SPR Analysis

1. Buffer preparation: Two-fold serial dilution of Analyte from 4000 nM to 15.625 nM (15.625, 31.25, 62.5, 125, 250, 500, 1000, 2000, 4000 nM).\
2. Chip running:
Contact time: 90 seconds
Dissociation time: 200 seconds
Flow rate: 50 mL/min.
3. Chip regeneration:
Buffer: Glycine solution (pH 2.0)
Contact time: 60 seconds
Flow rate: 50 mL/min.

Table 2 listed the binding affinity of five anti-Globo H antibodies (OBI-888, 82V, R783, R725-2 and R643). The binding affinity of low pI variants is reduced at the range of 4× by SPR. The binding constant (K_{D}) value were ranging for 9.751E-7 to 2.492E-7 M. However, the R643 antibody exhibited the lowest K_{D} value (9.751E-7).

TABLE 2
Binding affinity of five anti-Globo H antibodies assessed by SPR

| Sample | ka (1/Ms) | kd (1/s) | K_{D} (M) | Rₘₐₓ (RU) | Chi² (RU²) |
|---|---|---|---|---|---|
| OBI-888 | 1.594E+5 | 0.03971 | 2.492E−7 | 29.91 | 0.774 |
| 82V | 1.630E+5 | 0.04066 | 2.494E−7 | 29.40 | 0.600 |
| R783 | 4.270E+4 | 0.03577 | 8.377E−7 | 19.28 | 0.155 |
| R643 | 3.662E+4 | 0.03571 | 9.751E−7 | 12.75 | 0.117 |
| R725-2 | 4.090E+4 | 0.03470 | 8.484E−7 | 17.60 | 0.156 |

### Example 3. Pharmacokinetic Study of Anti-Globo H Antibody in Mouse

### (1) Animal and Study Design

82V, OBI-888 and R783 anti-Globo H antibodies were prepared in a formulation buffer (25 mM Na-citrate, 100 mM NaCl, pH6.5). The antibody preparation and administration were performed by Pharmacology Discovery Services, Taiwan. Female nude (nu/nu) mice, aged 6-7 weeks, were obtained from BioLASCO Taiwan Co. Ltd. The mice were divided into three groups based on the administered antibody. Each group contains two mice, and each mouse was injected with a specific anti-Globo H antibody intravenously on Day 1. Blood samples were collected at 1 h, 4 h, 8 h, Day 2, Day 4, Day 8, Day 15, Day 22, Day 29, Day 36 and Day 43 post antibody administration and stored at −80° C. Table 3 listed the pharmacokinetic (PK) study design detail in mice.

TABLE 3
Animal study (mouse) design of Anti-Globo H antibodies

| | | | Sample collection time points | | | | | | | | | | |
|---|---|---|---|---|---|---|---|---|---|---|---|---|---|
| | Number | | (Time post-dose) | | | | | | | | | | |
| Test | of | Dose | Day 1 | Day 1 | Day 1 | Day | Day | Day | Day | Day | Day | Day | Day |
| Article | animals | (mg/kg) | 1 hr | 4 hr | 8 hr | 2 | 4 | 8 | 15 | 22 | 29 | 36 | 43 |
| OBI- | 2 | 3 | ▪ | | ▪ | | ▪ | | ▪ | | ▪ | ● | |
| 888 | 2 | | | ▪ | | ▪ | | ▪ | | ▪ | | ● | |
| R783 | 2 | 3 | ▪ | | ▪ | | ▪ | | ▪ | | ▪ | | #● |
| | 2 | | | ▪ | | ▪ | | ▪ | | ▪ | | ▪ | #● |
| 82V | 2 | 3 | ▪ | | ▪ | | ▪ | | ▪ | | ▪ | ● | |
| | 2 | | | ▪ | | ▪ | | ▪ | | ▪ | | ● | |

▪: Non-terminal blood collections
●: Sacrifice and drain all the blood
#●: Sacrifice and drain all the blood: extended days to sacrifice

### (2) ELISA Assay

The ELISA assay procedure was summarized below. The calibration standards of the OBI-888 antibody and the R783 antibody were prepared in mouse serum matrix from 2500 ng/mL to 25 ng/mL. Biocheck kit (Cat. No. BC-5001) was used in the ELISA assay. Globo H ceramide coated plates were used as the capture antigen. Fifty microliter of 10-fold diluted calibration standard or test sample were added into each well and incubated at 25±2° C., 750 rpm for 45 minutes. Each well was then washed five times with Wash Buffer and 50 μL of goat Anti-Human IgG-HRP conjugate reagent was added to each well, then incubated at 25±2° C., 750 rpm for 45 minutes. Following incubation, 3,3′,5,5′-Tetramethylbenzidine (TMB) reagent was added to the wells and incubated at 25° C. for 15 minutes. 1N HCl was added as stop solution. Finally, the absorbance was read with a microtiter plate reader at 450 nm spectrum within 15 minutes of stop solution addition.

### (3) Non-Compartmental Analysis

PK parameters were estimated with Phoenix pharmacokinetic software (Winnonlin 8.1 Certara, USA), using a non-compartmental approach consistent with the IV bolus route of administration. All PK parameters were generated from individual concentrations of the 82V antibody, OBI-888 antibody and R783 antibody were estimated using nominal sampling times relative to the start of each dose administration. Concentration values reported as not quantifiable were assigned a value of zero. The area under the concentration (AUC) vs. time curve was calculated using the linear trapezoidal method with linear interpolation. The AUC was not calculated for PK profiles with less than three quantifiable concentrations of the 82V antibody, OBI-888 antibody and R783 antibody at separate time points. When practical, the terminal elimination phase of each concentration versus time curve was identified using at least the final three observed concentration values. The slope of the terminal elimination phase was determined using log linear regression on the unweighted concentration data. Parameters relying on the determination of the terminal elimination phase were not reported if the coefficient of determination was less than 0.800, or if the extrapolation of the AUC to infinity represented more than 20% of the total area. Table 4 showed the studied PK parameters of the 82V antibody, OBI-888 antibody and R783 antibody in mice.

TABLE 4
Pharmacokinetic parameters (mouse)
tested of the 82V, OBI-888 and R783 antibody

| Parameter | Description of Parameter |
|---|---|
| Tₘₐₓ | The time after dosing at which the maximum |
| | observed concentration was observed. |
| Cₘₐₓ | The maximum observed concentration measured |
| | after dosing. |
| AUC₍₀₋₁₎ | The area under the concentration versus time |
| | curve from the start of dose administration to |
| | the time after dosing at which the last quantifiable |
| | concentration was observed, using the linear |
| | trapezoidal method. |
| T_{1/2} | The apparent terminal elimination half-life. |
| CL | The apparent clearance rate of OBI-888 and R783 |
| | from the analyzed matrix. |
| Vd | The apparent volume of distribution of |
| | OBI-888/R783 in the mouse. |

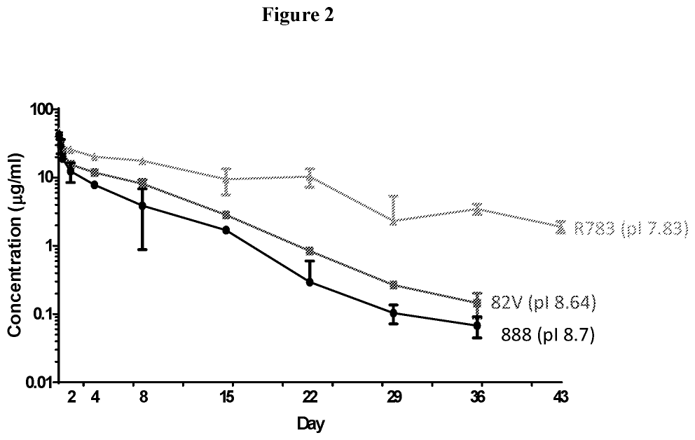

FIG. 2 and Table 5 illustrated the result of pharmacokinetic study in mouse. It indicated significant improvement of PK profile of the pI value lowered variant R783 antibody with the half life at 11.68 days (T_{1/2}=280.4 hours). It further demonstrated a lower pI value improves the stability of the anti-Globo H antibodies.

TABLE 5
Pharmacokinetic result (mouse) tested
of the 82V, OBI-888 and R783 antibody

| | Tₘₐₓ | T_{1/2} | Cₘₐₓ | AUC₀₋ₜ | Cl | Vd |
|---|---|---|---|---|---|---|
| Antibody | (h) | (h) | (μg/mL) | (h × μg/mL) | (mL/h/kg) | (mL/kg) |
| 82V | 1 | 115.15 | 41.16 ± | 3561.9 ± | 0.84 | 138.98 |
| | | | 0.48 | 142.08 | | |
| R783 | 1 | 280.4 | 46.76 ± | 9620.3 ± | 0.29 | 116.77 |
| | | | 1.14 | 1040.99 | | |
| 888 | 1 | 100.39 | 42.35 ± | 2244.7 ± | 1.33 | 192.72 |
| | | | 0.55 | 419.88 | | |

### Example 4. Pharmacokinetic Study of Anti-Globo H Antibodies in Monkey

### (1) Animal and Study Design

The 82V, OBI-888 and R783 anti-Globo H antibodies were prepared in a formulation buffer (25 mM Na-citrate, 100 mM NaCl, pH6.5). Each assay plate included calibration standards, quality control samples and blank matrix. Each sample was analyzed in duplicate and the mean value of the two replicates was reported. Samples were subject to a minimum dilution of 1 in 20 in assay buffer. Samples requiring a dilution greater than 1 in 20 were further diluted in Sample Dilution Buffer. Table 6 listed the PK study design in monkey.

TABLE 6
Animal study (monkey) design of Anti-Globo H antibodies

| | |
|---|---|
| Number of | Total of 3 monkeys, one male for group 1 |
| Monkey | (the OBI-888 antibody) and one male and |
| | one female for group 2 (the R783 antibody). |
| Body Weight | 1) Recorded before dosing on day 1. |
| Frequency | 2) Each dose group record once weekly as |
| | well as on the day of sacrifice and until |
| | study termination. |
| Dose | The OBI-888 antibody (group 1) and the |
| Administration | R783 antibody (group 2) |
| Dose Route | Slow IV bolus, over approximately 6 min |
| Dose Frequency | Single dose |
| Time Points | PK Sampling time: Pre-dose, 1, 4 h, 8 h, 24 h, |
| | 3 d (72 h), 7 d (168 h), 14 d (336 h), 21 d (504 h), |
| | 28 d (696 h) 35 d (864 h) and 42 d (1032 h) post dose |
| | Note: Timing after the completion of IV bolus |
| | administration |
| Collection Site | Femoral vein |
| Blood Volume | Approximately 1 mL/time point. |
| Anticoagulant | SST tubes to hold blood for serum samples. |
| Handling after | Blood was collected into serum separation |
| Collection | tubes (SST) and held at room temperature |
| | for at least 30 minutes and allowed to clot |
| | prior to centrifugation. Blood sample was |
| | centrifuged at 2 to 8° C. for approximately |
| | 10 minutes at approximately 2700 g within |
| | 2 hours of collection, and serum was harvested. |
| | About 0.25 mL of processed serum sample |
| | was transferred to bioanalysis tube and stored at |
| | −60 to −80° C. immediately and until transferred |
| | to the Covance-Shanghai Metabolism group. |
| Transfer | The serum samples from the monkeys in |
| Conditions | the OBI-888 antibody group and the R783 |
| | antibody group were transferred to Covance- |
| | Shanghai Metabolism group with dry ice. |
| Storage | All serum samples were stored in a freezer, |
| Conditions | set to maintain −60 to −80° C., until analyzed. |
| Storage of | The serum samples were maintained at |
| remaining | Covance Shanghai in a freezer set to |
| samples | maintain −60 to −80° C. for at least 90 days. |
| Termination | All surviving animals were transferred to |
| | stock colony within 24 hours after last |
| | blood collection. Animals that died or |
| | euthanized at moribundity were discarded |
| | without necropsy. |

### (2) ELISA Assay

The ELISA assay procedure was summarized below. The calibration standards of the OBI-888 antibody and R783 antibody were prepared in mouse serum matrix from 2500 ng/mL to 25 ng/mL. Biocheck kit (Cat. No. BC-5001, BIO-CHECK Laboratories Ltd.,) was used in the ELISA assay. Globo H ceramide coated plates were used as the capture antigen. Fifty microliter of 10-fold diluted calibration standard or test sample were added into each well and incubated at 25±2° C., 750 rpm for 45 minutes. Each well was then washed five times with Wash Buffer; 50 μL of goat Anti-Human IgG-HRP conjugate reagent was added to each well, then incubated at 25±2° C., 750 rpm for 45 minutes. Following incubation, 3,3′,5,5′-Tetramethylbenzidine (TMB) reagent was added to the wells and incubated at 25° C. for 15 minutes. 1N HCl was added as the stop solution. Finally, the absorbance was read with a microtiter plate reader at 450 nm spectrum within 15 minutes of stop solution addition.

### (3) PK Parameter Calculation

Sample results were accepted if calibration curve and QC data from the reported batches indicated that the method met the acceptance criteria for those batches. 75% of standards within the quantifiable range (LLOQ to ULOQ) must be included (minimum of 6 non-zero calibration standards). 25% may be masked from the curve. A calibration point with acceptable % CV and % Bias may not be masked to enable the calibration curve to compute the concentration of samples or controls within target acceptance limits. Two duplicates of each LQC, MQC and HQC level will be assessed in each analysis batch. At least 67% of all QC samples, with ≥50% at each level must generate a result within ±20% bias of the nominal value and precision between sample replicates of ≤20%. Precision (CV %) between sample replicates should not exceed 20% based upon concentration. This does not apply to samples falling below the LLOQ of the assay. Table 7 listed the studied PK parameters and the calculation methods of the OBI-888 antibody and R783 antibody

TABLE 7
Pharmacokinetic parameters (monkey) tested
of OBI-888 antibody and R783 antibody

| | |
|---|---|
| T_{1/2} | Elimination half-life |
| Cₘₐₓ | The maximum of serum concentration |
| Tₘₐₓ | The time of peak concentration |
| AUC₀₋ₗₐₛₜ | Area under the concentration-time curve |
| | from Hour 0 to the last time point with |
| | measurable concentrations |
| AUC_{0-inf} | Area under the concentration-time curve |
| | from hour 0 to infinity, estimated by linear |
| | trapezoidal rule |
| CL | Clearance |
| MRT | Mean residence time |
| Vd | Apparent volume of distribution |

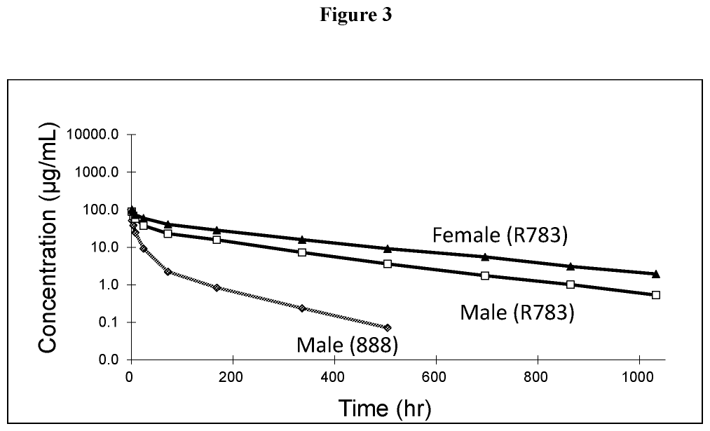

FIG. 3 and Table 8 illustrated the result of pharmacokinetic study in monkey. It indicated that pI value lowered variant R783 antibody with a significant improvement of PK profile in monkey, such as a longer half life of 8.88 days (T_{1/2}=213.1 hours). It further demonstrated a lower pI value improves the stability of anti-Globo H antibodies.

TABLE 8
Pharmacokinetic result (monkey) tested of OBI-888 antibody and R783 antibody

| | Dose | | | | | | | | |
|---|---|---|---|---|---|---|---|---|---|
| | Level | | Tmax | Cmax | C₀ | T_{1/2} | AUC₀₋ₗₐₛₜ | Vd | C1 |
| Antibody | (mg/kg) | Gender | (h) | (μg/mL) | (μg/mL) | (day) | (h × μg/mL) | (mL/kg) | (mL/day/kg) |
| OBI-888 | 3 | Male | 1.0 | 51.7 | 57.1 | 3.95 | 1125 | 361 | 63.4 |
| R783 | 3 | Male | 1.0 | 89.3 | 97.3 | 8.06 | 8394 | 98.0 | 8.43 |
| | | Female | 1.0 | 101 | 106 | 9.66 | 15866 | 60.8 | 4.36 |
| | | Mean | 1.0 | 95.2 | 102 | 8.88 | 12100 | 79.4 | 6.38 |

A comparison of the pharmacokinetics parameters in mice and monkey of Herceptin, Perjeta, the OBI-888 antibody and R783 antibody was conducted, as these antibodies can be used to treat human patient with breast cancer. Table 9 listed the half life (T_{1/2}) of the aforementioned antibodies. The clearance and half-life of R783 was better than that of Herceptin and close to that of Perjeta.

TABLE 9
Pharmacokinetic parameters of Herceptin, Perjeta, the OBI-888 antibody and R783 antibody

| | Parameters | | | | | | | |
|---|---|---|---|---|---|---|---|---|
| | Herceptin^{a} | | Perjeta^{b} | | OBI-888 | | R783 | |
| | Species/strain | | | | | | | |
| | Mouse/ | Monkey/ | Mouse/ | Monkey/ | Mouse/ | Monkey/ | Mouse/ | Monkey/ |
| | C57BL6 | Cyno | CD-1 | Cyno | Nu/Nu | Cyno | Nu/Nu | Cyno |
| Dose (mg/kg) | 4 | 5 | 3, 30, 90 | 15, 50, 150 | 5 | 10, 100, 300 | 3 | 3 |
| Route | IV | IV | IV | IV | IV | IV | IV | IV |
| T_{1/2} (d) | 9.3 ± 1.1 | 5.2 ± 1.5 | 11.4-15.7 | 9.9-10.4 | 5.1 | 1.2-1.6 | 11.7 | 8.88 |
| Cl (ml/d/kg) | 10.3 ± 2.1 | 8.2 ± 3.1 | 5.6-9.2 | 5.0-5.2 | 29.6 | 33.6-43.4 | 6.96 | 6.38 |
| Vₛₛ (ml/kg) | 130 ± 19 | 59 ± 19 | 102-148 | 68-73 | 177.3 | 64-79 | 101.18 | 79.2 |

^{a}Result listed in reference: Ningyan et al., (2011) mAbs, 3(3): 289-298.
^{b}Result listed in reference: Camellia et al., (2006) Cancer Immunol Immunother, 55(6): 717-727.

### Example 5. Efficacy Study of Anti-Globo H Antibody in MCF-7 Xenograft Model

### (1) Cell Line and Culture Media

Human breast adenocarcinoma tumor cells, MCF-7 (1×10⁸ cells/mL) were froze and cultured in the lab of Pharmacology Discovery Services Taiwan, Ltd. MCF-7 tumor cell inoculum containing 2×10⁷ cells (0.2 mL mixture of matrigel and complete medium; 1:1) was implanted subcutaneously in the right flank of each mouse.

### (2) Animal

Female (nu/nu) nude mice aged 5-6 weeks obtained from BioLasco Taiwan (under Charles River Laboratories Licensee) were used. The animals were housed in individually ventilated cages (IVC, 36 Mini Isolator System). The allocation for five animals was 27×20×14 cm³. All animals were maintained in a hygienic environment under controlled temperature (20-24° C.) and humidity (30-70%) with 12-hour light/dark cycle. Free access to standard lab diet [MFG (Oriental Yeast Co., Ltd., Japan)] and autoclaved tap water were granted. All aspects of this work including housing, experimentation, and animal disposal were performed in general accordance with the “Guide for the Care and Use of Laboratory Animals: Eighth Edition” (National Academies Press, Washington, D.C., 2011) in our AAALAC-accredited laboratory animal facility. In addition, the animal care and use protocol was reviewed and approved by the IACUC at Pharmacology Discovery Services Taiwan, Ltd.

### (3) Reagent and Equipment

Reagent: β-Estradiol 3-benzoate (Sigma-Aldrich, Cat. No. E8515), FBS, insulin, L-glutamine, MEM, Matrigel Matrix (Corning), PBS, Penicillin streptomycin, Sodium pyruvate and Trypsin. Equipment: Biosafety cabinet (BSC) (NUAIR), Calipers (Mitutoyo), Centrifuge Himac CT6D (HITACHI), Centrifuge 5810R (Eppendorf), CO₂ Incubator (SANYO), Individually Ventilated Cages (36 Mini Isolator system), Inverted Microscope CK-40 (Olympus), Mouse Scale (TANITA), Vertical laminar flow (Tsao-Hsin, Taiwan) and Water bath (DEAGLE, Taiwan)

### (4) Method

Female athymic (nu/nu) nude mice, 5-6 weeks old, were used as detailed in the preceding section. Viable MCF-7 cells (obtained from OBI) were subcutaneously (SC) implanted (2×10⁷ cells/mouse with complete medium and matrigel (1:1) at 0.2 mL/mouse) into the right flank of female nu/nu mice. Thirteen days post tumor cell implantation; tumor bearing mice were divided into seven treatment groups, each group containing ten animals when group mean tumor volumes reached approximately 174 mm3 (denoted as Day 1). Supplemental βEstradiol 3-benzoate (100 μg/mouse) was injected subcutaneously into all mice twice weekly, starting one week before cell implantation, and continuing through the study period.

In study group 1, vehicle (25 mM sodium citrate, 100 mM NaCl pH6.5) was administered intravenously (IV) twice weekly for four weeks (eight total administrations) in a dose volume of 10 mL/kg. Study group 1 served as the negative control for comparing anti-tumor effect of test substance treated groups 2-7. In study group 2, test substance, OBI-888 antibody at 30 mg/kg, was administered intravenously (IV) once weekly for four consecutive weeks in a dose volume of 10 mL/kg. In study group 3, test substance, OBI-888 antibody at 30 mg/kg, was administered intravenously (IV) twice weekly for four consecutive weeks (eight total administrations) in a dose volume of 10 mL/kg. In study group 4, test substance, R783 antibody at 30 mg/kg, was administered intravenously (IV) once weekly for four consecutive weeks in a dose volume of 10 mL/kg. In study group 5, test substance, R783 antibody at 30 mg/kg, was administered intravenously (IV) twice weekly for four consecutive weeks (eight total administrations) in a dose volume of 10 mL/kg. In study group 6, test substance, R725-2 antibody at 30 mg/kg, was administered intravenously (IV) once weekly for four consecutive weeks in a dose volume of 10 mL/kg. In study group 7, test substance, R725-2 antibody at 30 mg/kg, was administered intravenously (IV) twice weekly for four consecutive weeks (eight total administrations) in a dose volume of 10 mL/kg.

In-life blood samples were collected from all experimental mice prior to tumor cell implantation, and on Day 8 and Day 22 prior to dose administrations. Upon study completion, terminal blood samples were collected from all mice. All blood samples were processed to serum, flash frozen, and stored at −80° C. Upon study completion, tumor samples were harvested from all remaining animals on study. The tumor masses were cut in half and either placed in 10% formalin and stored at room temperature, or flash frozen and stored at −80° C. The tumor volume, body weight, mortality, and signs of over toxicity were monitored and recorded twice weekly for 29 days. Tumor volume (mm³) was estimated according to the ellipsoid formula as: Length×(Width)²×0.5. Tumor growth inhibition (% T/C) was calculated by the following formula: T/C=(Tn/Cn)×100% Cn: Tumor weight measured on Day n in the control group Tn: Tumor weight measured on Day n in the treated group T/C value≤42% was considered significant antitumor activity (#). Percent Tumor Growth Inhibition (% TGI) was also calculated by the following formula: % TGI=(1−[(T−T0)/(C−C0)])×100 T: Mean tumor volume of treated group T0: Mean tumor volume of treated group at study start C: Mean tumor volume of control group C0: Mean tumor volume of control group at study start Two-way ANOVA followed by Bonferroni post-tests were also applied to ascertain the statistical significance between the vehicle and test substance-treated groups. Differences are considered significant at p<0.05 (*).

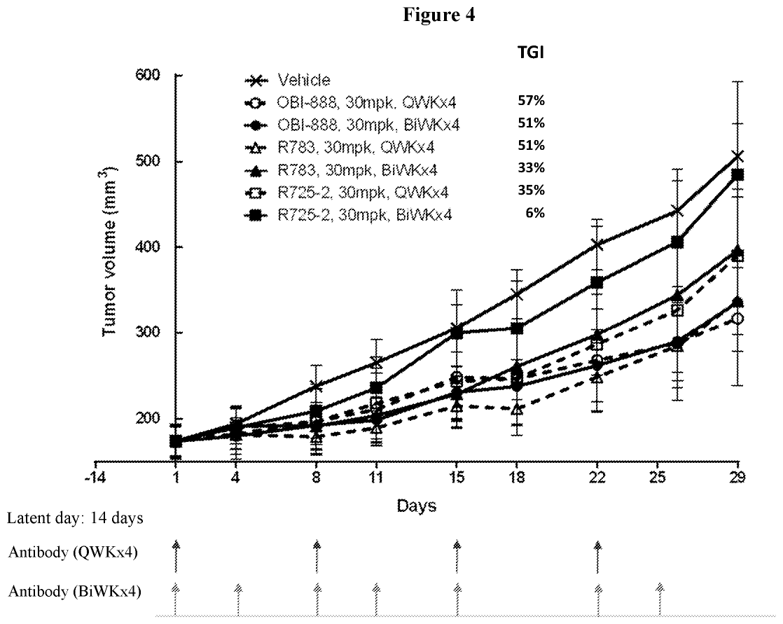

FIG. 4 illustrated the efficacy of the OBI-888 antibody, R783 antibody, and R725-2 antibody in inhibiting the MCF-7 cells. The Tumor Growth Inhibition (% TGI) values were: 57% (once injection weekly) and 51% (twice injection weekly) for OBI-888 antibody, 35% (once injection weekly) and 6% (twice injection weekly) for R725-2 antibody, and 51% (once injection weekly) and 33% (twice injection weekly) for R783 antibody. The results indicated that both R783 antibody and R725-2 antibody were more effective in inhibiting tumor cells than the OBI-888 antibody.

### Example 6. NK Cell-Based ADCC (Antibody-Dependent Cell-Mediated Cytotoxicity) of Anti-Globo H Antibody

### (1) Reagent

NK Cell Isolation Kit human (MACS, Cat. No. 130-092-657)
NK MACS® Medium human (MACS, Cat. No. 130-114-429)
Human AB serum (Sigma-Aldrich, Cat. No. H3667)
Human IL-2 IS premium grade (MACS, Cat. No. 130-097-745)
NK Cell Activation/Expansion Kit (MACS, Cat. No. 130-094-483)
MACS Rinsing buffer (MACS, Cat. No. 130-091-022)
PBMC (Lonza, Cat. No. CC-2702): Thawed PBMC in 3 mL Rinsing buffer and centrifuged with 300 g for 5 minutes. Removed supernatant solution and repeated three times.
LS Column (MACS, Cat. No. 130-042-401)
NutriFreez™ D10 Cryopreservation Medium (Biological Industries, Cat. No. 05-713-1B)
RRMI-1640 (Gibco, Cat. No. A10491-01)
FBS (Gibco, Cat. No. 16000044)
Bio-Glo (Promega, Cat. No. G7941)

### (2) Buffer Preparation

Binding buffer: 0.5% FBS in MACS Rinsing buffer
NK culture medium: NK MACS® Medium+Human IL-2 IS premium grade+5% human AB serum
NK Cell Activation/Expansion beads (final stock: 1E5 beads/μL):
100 μL of CD335 (NKp46)-Biotin+100 μL CD2-Biotin+500 μL Anti-Biotin MACSiBead Particles+300 μL PBS (total 1 mL in 2 mL tube) and incubate for 2 hours at 2-8° C. under rotation by using the rotator at approximately 4 rpm.
Seeding medium: 10% FBS in RRMI-1640

### (3) Plating Target Cell

1. Dispense 100 μL of HCC1428 culture medium into those outermost wells of white 96-well assay plate which labeled “B” in the plate layout of attachment.
2. Pipet 6.65 mL of HCC1428 culture medium in multi-channel solution basin.
3. Remove 1 vial of HCC1428 cells from liquid nitrogen tank. Thawed vial in a 37° C. water bath until cells are thawed. Do not invert.
4. Pipet 350 μL of the thawed HCC1428 cells into multi-channel solution basin prepared in step 2.
5. Dispensed 100 μL of MCF7W cells to the inner 60 wells of white 96-well assay plate prepared in step 1 using a multichannel pipette. Each well contains 2.5×10⁴ HCC1428 cells. Placed lid on the plate and incubated overnight in a CO₂ incubator at 37° C.

### (4) Preparing Antibody Serial Dilutions

1. Prepared a sterile clear V-bottom 96-well plate for preparing antibody serial dilutions. For threefold serial dilutions, performed the dilutions.
2. The tested antibody was diluted with ADCC assay buffer to make a concentration at 2× dilution.
3. Added 100 μL of antibody starting dilution (dilu1, 2× final concentration) to column 11.
4. Added 100 μL of ADCC assay buffer to other wells from column 10 to column 2.
5. Used a multichannel pipette, transfer 50 μL from the antibody starting dilutions in column 11 into column 10. Mixed well by pipetting and avoided creating bubbles.
6. Repeated equivalent threefold serial dilutions across columns from right to left until column 3. Note: Column 2 contained 100 μL of ADCC assay buffer as a “no-antibody” control.
7. Transferred the MCF7W culture plate from CO₂ incubator to cabinet. Aspired HCC1428 culture medium from inner 60 wells.
8. Transferred 37.5 μL of the antibody dilution series to the indicated wells in the HCC1428 culture plate using a multichannel pipette.
9. Placed the plate in the cabinet during preparation of target cells at the next step.

### (5) Plating Effector Cell

1. Pipetted 2700 μL of ADCC assay buffer in multi-channel solution basin.
2. Removed 1 vial of ADCCECW cells from liquid nitrogen tank. Thawed vial in a 37° C. water bath until cells are thawed.
3. Pipetted 300 μL of the thawed ADCCECW cells into multi-channel solution basin.
4. Dispensed 37.5 μL of ADCCECW cells to the inner 60 wells of assay plate using a multichannel pipette. Each well contained 7.5×10⁴ ADCCECW cells. Placed lid on the plate and incubated for 5.75 hours in a CO₂ incubator at 37° C.

### (6) Negative Selection of CD56 Positive NK Cells

1. Added NK cell biotin antibody cocktail (from NK Cell Isolation Kit) into darked tube for binding 20 minutes (Dilution fold: 1E8 cells=100 μL antibody+400 μL Binding buffer; 9E7 cells=90 μL antibody+360 μL Binding buffer; 8E7 cells=80 μL antibody+320 μL Binding buffer).
2. Added NK cell microbeads cocktail (from NK Cell Isolation Kit) into darked tube for binding 20 minutes (Microbeads: Antibody=2:1).
3. Added binding buffer and centrifuged with 300 g for 5 minutes. Removed supernatant solution and repeated three times.
4. Used 3 mL binding buffer to re-suspend cell pellet and added into column (NK culture density: 1E6/mL, NK Cell Activation/Expansion beads-to-cell ratio 1:2).

### (7) Primary ADCC by CD56 Positive NK Cells

1. Seeded HCC-1428-Luc (or NCI-N87-Luc) 2E4 cells/well with 100 μL seeding medium in 96-well pate and cultivated in 37° C., 5% CO₂ overnight.
2. Added 2× concentration antibody with 50 μL seeding medium into 96-well plate.
3. Removed NK Anti-Biotin MACSiBead particles by MACSiMAG™ separator and centrifuged with 300 g for 5 minutes.
4. Re-suspend the cell pellet with seeding medium and counted cell number.
5. Adjusted the concentration of effector cell. Then added 50 μL cell into the 96-well plate filled with antibody.
6. After incubated 24 hours in 37° C., 5% CO₂, medium was replaced by 70 μL fresh seeding medium.
7. Added 70 μL bio-glo into 96-well plate and Shaked 15 minutes to lysis target cell.

### (8) Adding Bio-Glo Luciferase Assay Reagent

1. Thawed one tube of the Bio-Glo Luciferase Assay Reagent to ambient temperature 2 hours before added into the assay plate.
2. Removed assay plates from the 37° C. incubator and equilibrated to ambient temperature on the bench for 15 minutes.
3. Added 75 μL of Bio-Glo Luciferase Assay Reagent to all the inner 60 wells of the assay plates using a manual multichannel pipette and avoided creating any bubbles.
4. Covered the plate with aluminum foil and incubated at ambient temperature for 15 minutes.
5. Measured luminescence using luminometer microplate reader, SpectraMax L.

### (6) Data Analysis

1. Calculated the average relative light unit (RLU) from no antibody control wells, B2 to G2.
2. Calculated the fold of induction=RLU (induced)/RLU (average of no antibody control).
3. Graphed data as RLU Fold of Induction versus Log10 [antibody]. Fitted curves with 4-parameter logistic model and determine EC₅₀ and relative potency of antibody response using PRISM 6.0 and Gen5 Microplate Reader and Imager Software, respectively.

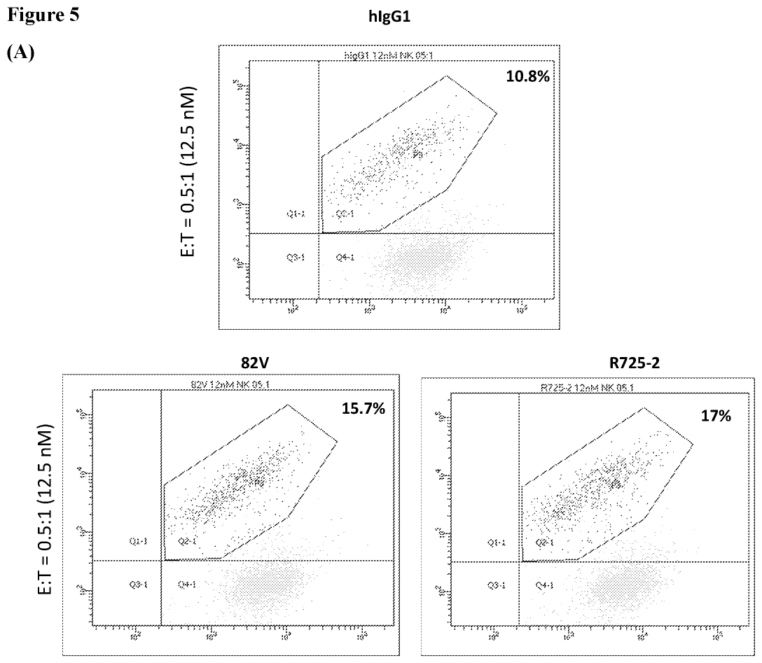

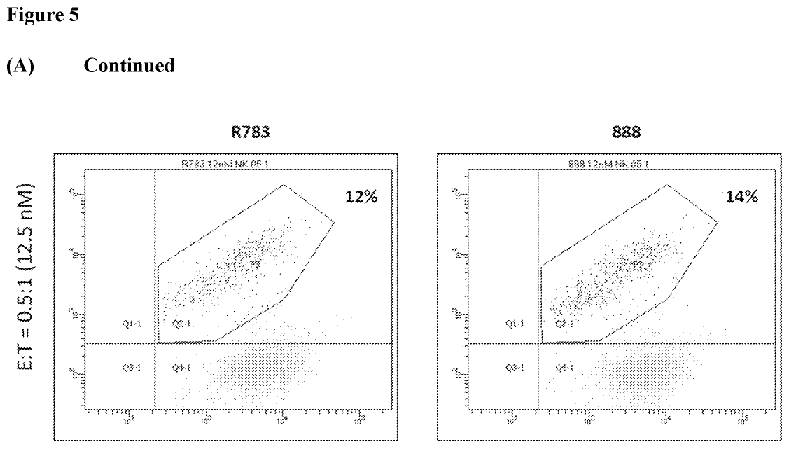

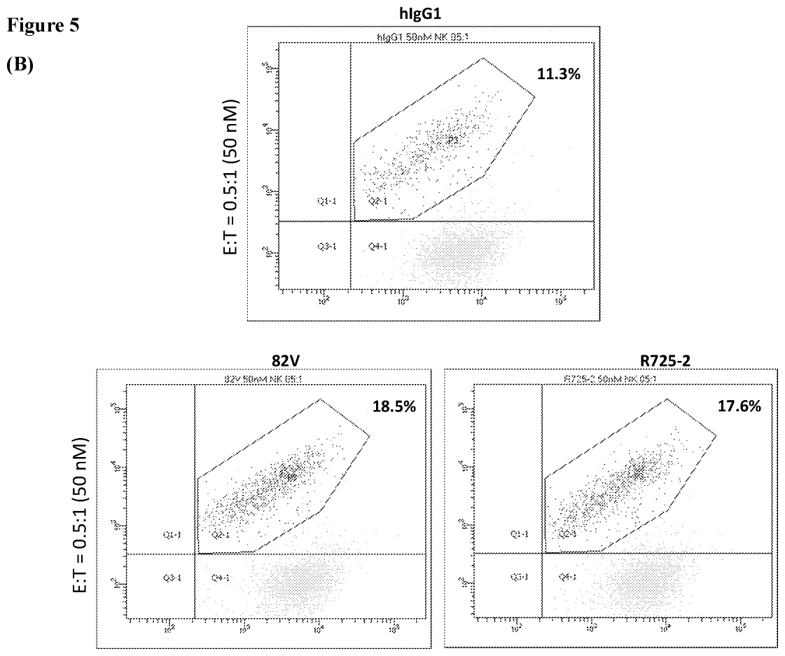

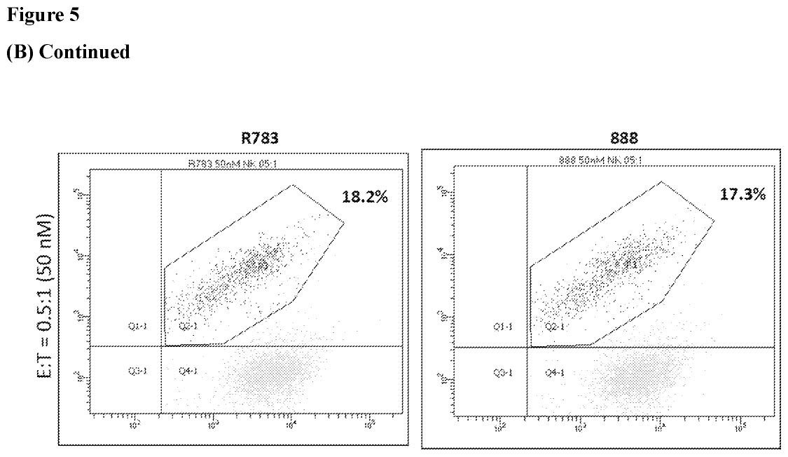

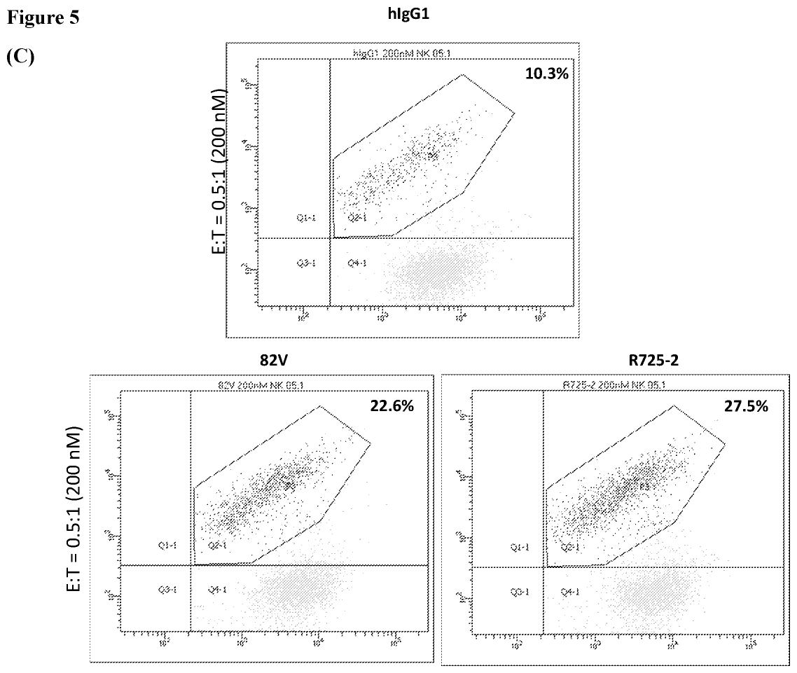

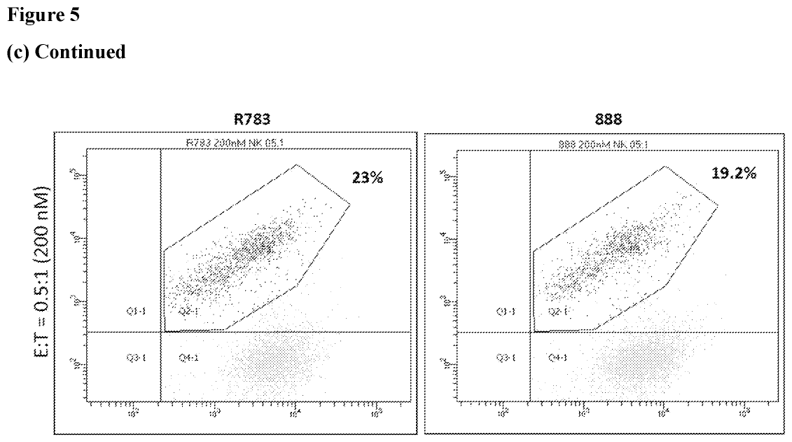

FIG. 5 and Table 10 illustrated the cytotoxicity of the OBI-888 antibody, 82V antibody, R783 antibody and R725-2 antibody using CD56+ NK cell. A dose-dependent NK cell-based ADCC in the OBI-888 antibody, 82V antibody, R783 antibody and R725-2 antibody was noted (Panel A: 12.5 nM, Panel B: 50 nM; Panel C: 200 nM). The 82V antibody, R783 antibody and R725-2 antibody displayed better NK cell-based ADCC activity than OBI-888 antibody.

TABLE 10
NK cell-based ADCC activity of 82V,
R725-2, R783 and OBI-888 antibodies

| Concentration (nM) | 82V | R725-2 | R783 | OBI-888 |
|---|---|---|---|---|
| 12.5 | 4.1% | 5.3% | 0.2% | 2.2% |
| 50 | 6.4% | 5.3% | 6.0% | 4.9% |
| 200 | 11.4% | 16% | 11.8% | 7.7% |

### Example 7. ADCP (Antibody-Dependent Cellular Phagocytosis) of Anti-Globo H Antibody

### (1) Reagent

CD14 Microbeads human (MACS, Cat. No. 130-050-201)
MACS Rinsing buffer (MACS, Cat. No. 130-091-022)
PBMC (Lonza, Cat. No. CC-2702): Thawed PBMC in 3 mL Rinsing buffer and centrifuged with 300 g for 5 minutes. Removed supernatant solution and repeated three times.
LS Column (MACS, Cat. No. 130-042-401)
RRMI-1640 (Gibco, Cat. No. A10491-01)
FBS (Gibco, Cat. No. 16000044)
Recombinant Human M-CSF (Peprotech, Cat. No. 300-25)
Bio-Glo (Promega, Cat. No. G7941)
CellTracker™ Green CMFDA Dye (Invitrogen, Cat. No. C7025)
CellTracker™ Deep Red (Invitrogen, Cat. No. C34565)
Cellstripper (Corning, Cat. No. 25-056-CI)

### (2) Buffer Preparation

Binding buffer: 0.5% FBS in MACS Rinsing buffer
MCSF stock (100 ng/μL): 100 μg MCSF powder+1 mL serum free RRMI-1640
Macrophage culture medium:
20% FBS in RRMI-1640+100 ng/mL MCSF
10% FBS in RRMI-1640+50 ng/mL MCSF
Assay medium: 1% FBS in RPMI-1640:
Prepare CellTracker™ Green and CellTracker™ Deep Red:
Dissolved CellTracker™ Green in DMSO to a final concentration of 10 mM 50 μg in 10.8 μL DMSO.
Dissolved CellTracker™ Deep Red in 20 μL DMSO per vial to make a 1 mM (1000×) solution.

### (3) Positive Selection of CD14 Monocyte

1. Added CD14 beads into darked tube with binding buffer for binding 20 minutes (1E7 cells 15 μL CD14 beads+35 μL Binding buffer=50 μL; 2E7 cells 30 μL CD14 beads+70 μL Binding buffer=100 μL; 3E7 cells 45 μL CD14 beads+105 μL Binding buffer=150 μL).
2. Added binding buffer and centrifuged with 300 g for 5 minutes. Removed supernatant solution and repeated three times.
3. Used 3 mL binding buffer to re-suspend cell pellet and added into column (Macrophage culture density: 2E6/2 mL per well in 6 well plate; Monocyte:PBMC=1:10 to 1:20).

### (4) Primary ADCP by M0 Macrophage Cells

1. CellTracker™ Green stain Target cell (HCC-1428)
2. Added 1E7 cells in 1 mL serum free RPMI-1640 (10000× dilution) for 37° C., 15 minutes.
3. Centrifuged with 300 g for 5 minutes. Removed supernatant solution and re-suspend cell pellet with 5 mL assay medium.
4. Added diluted antibody solution into cell medium for binding (Antibody in 100 μL Target cell+100 μL Effector cell).
5. Washed macrophage with PBS and added 500 μL/per well Cellstripper in 37° C. for 5 minutes.
6. Centrifuged with 300 g for 5 minutes. Removed supernatant solution and re-suspend cell pellet with serum free medium.
7. Added CellTracker™ Deep Red for effector cell (4E5 cells) staining (effector cell: Target cell=2:1).
8. Centrifuged with 300 g for 5 minutes. Removed supernatant solution and re-suspend cell pellet with 5 mL assay medium.
9. Added into the eppendorf with target cell+Antibody (Antibody in 100 μL Target cell+100 μL Effector cell).
10. Incubated for 3-4 hours in 37° C., 5% CO₂. Centrifuged with 300 g for 5 minutes and removed supernatant solution.
11. Fixed cell pellet for 20 minutes and analyzed by Flow Cytometry.

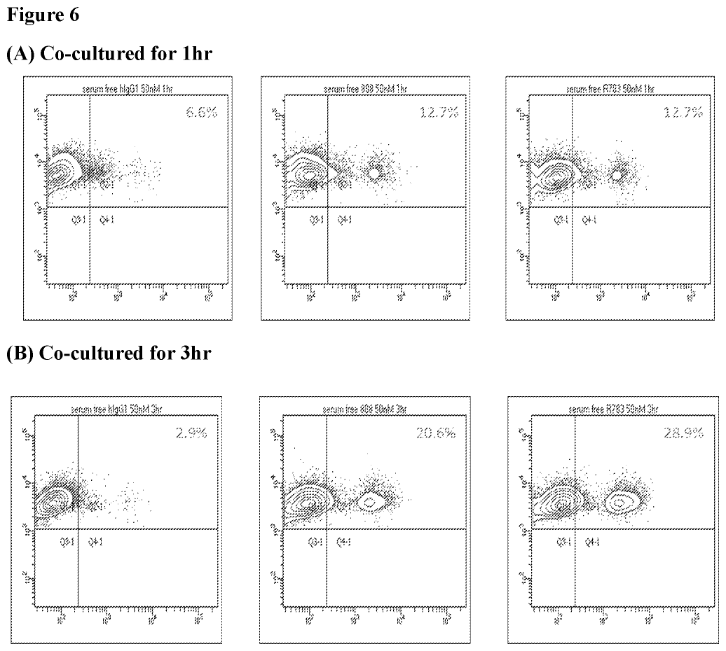

FIG. 6 illustrated the phagocytosis of the OBI-888 antibody and R783 antibody. Antibody pre-treated target cells and healthy donor macrophage were co-cultured in serum free medium for 1 hour (Panel A) and 3 hours (Panel B). It indicated that both OBI-888 antibody and R783 antibody promoted ADCP under suspension co-culture of E/T cells in serum free medium condition.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. An antibody or an antibody binding portion thereof that binds to Globo H, comprising a heavy chain variable region and a light chain variable region,
wherein the heavy chain variable region comprises three complementarity determining regions (CDRs), HCDR1, HCDR2 and HCDR3, having amino acid sequences set forth in SEQ ID NOs: 1, 2 and 3, respectively, and
wherein the light chain variable region comprises three CDRs, LCDR1, LCDR2 and LCDR3, having amino acid sequences set forth in SEQ ID NOs: 4, 5 and 6, respectively.

2. (canceled)

3. The antibody or the antigen binding portion thereof of claim 1, wherein the heavy chain variable domain comprising an amino acid sequence 90% to 100% identical to the amino acid sequence shown in SEQ ID NO: 19 and the light chain variable domain comprising an amino acid sequence 90% to 100% identical to the amino acid sequence shown in SEQ ID NO: 20.

4. An antibody or an antibody binding portion thereof that binds to Globo H, comprising a heavy chain variable region and a light chain variable region,
wherein the heavy chain variable region comprises three complementarity determining regions (CDRs), HCDR1, HCDR2 and HCDR3, having amino acid sequences set forth in SEQ ID NOs: 7, 8 and 9, respectively, and
wherein the light chain variable region comprises three CDRs, LCDR1, LCDR2 and LCDR3, having amino acid sequences set forth in SEQ ID NOs: 10, 11 and 12, respectively.

5. (canceled)

6. The antibody or the antigen binding portion thereof of claim 3, wherein the heavy chain variable domain comprising an amino acid sequence 90% to 100% identical to the amino acid sequence shown in SEQ ID NO: 21 and the light chain variable domain comprising an amino acid sequence 90% to 100% identical to the amino acid sequence shown in SEQ ID NO: 22.

7-8. (canceled)

9. An antibody or an antibody binding portion thereof that binds to Globo H, comprising a heavy chain variable region and a light chain variable region, wherein the heavy chain variable domain comprising an amino acid sequence 90% to 100% identical to the amino acid sequence shown in SEQ ID NO: 23 and the light chain variable domain comprising an amino acid sequence 90% to 100% identical to the amino acid sequence shown in SEQ ID NO: 24.

10. (canceled)

11. The antibody or the antigen binding portion thereof of claim 1, wherein the antibody or antigen-binding portion thereof is: (a) a whole immunoglobulin molecule; (b) an scFv; (c) a Fab fragment; (d) an F(ab′)₂; or (e) a disulfide link that specifically binds to a T cell surface antigen Fv.

12. The antibody, or an antigen-binding portion thereof of claim 6, wherein the antibody is a humanized antibody or a bi-specific antibody.

13. The antibody of claim 7, wherein the humanized antibody is an IgG or IgM.

14. (canceled)

15. The bi-specific antibody of claim 7, wherein the bi-specific antibody comprises a first binding domain and a second binding domain.

16. The bi-specific antibody of claim 9, wherein the first binding domain binds to a Globo series antigen and the second binding domain binds to a T cell surface antigen.

17. The bi-specific antibody of claim 10, wherein the Globo series antigen is Globo H, SSEA-3 or SSEA-4.

18. The bi-specific antibody of claim 10, wherein the T cell surface antigen is CD2, CD3, CD4, CD5, CD6, CD8, CD28, CD40L or CD44.

19-20. (canceled)

21. A method for inhibiting the proliferation of cancer cells, comprising the administering of an effective amount of an antibody or antigen-binding portion thereof of claim 1 to a patient, wherein the proliferation of cancer cells is inhibited.

22. (canceled)

23. The method of claim 13, wherein the cancer is a Globo H expressing cancer.

24. The method of claim 14, wherein the Globo H expressing cancer is selected from the group consisting of sarcoma, skin cancer, leukemia, lymphoma, brain cancer, glioblastoma, lung cancer, breast cancer, oral cancer, head-and-neck cancer, nasopharyngeal cancer, esophageal cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, and prostate cancer.

25. The method of claim 13, wherein the effective amount of an antibody or antigen-binding portion thereof is 0.01 mg to 10 g or 0.001 to 60 mg/kg body weight of the subject.


SEQUENCE LISTING (24 sequences)

SEQ ID NO: 1 - protein, 12 aa, Artificial Sequence
  R783 first heavy chain complementarity determining region (HCDR1)
GFSLYRFDMGVG

SEQ ID NO: 2 - protein, 19 aa, Artificial Sequence
  R783 second heavy chain complementarity determining region (HCDR2)
WDHIWWDDDKYYNPALKSR

SEQ ID NO: 3 - protein, 11 aa, Artificial Sequence
  R783 third heavy chain complementarity determining region (HCDR3)
VRGLHDYYYYF

SEQ ID NO: 4 - protein, 10 aa, Artificial Sequence
  R783 first light chain complementarity determining region (LCDR1)
CQASEDVSYM

SEQ ID NO: 5 - protein, 7 aa, Artificial Sequence
  R783 second light chain complementarity determining region (LCDR2)
YGTSNKA

SEQ ID NO: 6 - protein, 8 aa, Artificial Sequence
  R783  third light chain complementarity determining region (LCDR3)
CQQWSRRP

SEQ ID NO: 7 - protein, 12 aa, Artificial Sequence
  R725-2 HCDR1
GFSLYRFDMGVG

SEQ ID NO: 8 - protein, 17 aa, Artificial Sequence
  R725-2 HCDR2
HIWWDDDKYYNPALQSR

SEQ ID NO: 9 - protein, 11 aa, Artificial Sequence
  R725-2 HCDR3
VRGLHDYYYYF

SEQ ID NO: 10 - protein, 10 aa, Artificial Sequence
  R725-2 LCDR1
CQASEDVSYM

SEQ ID NO: 11 - protein, 7 aa, Artificial Sequence
  LCDR2
YGTSNKA

SEQ ID NO: 12 - protein, 8 aa, Artificial Sequence
  R725-2 LCDR3
CQQWSRRP

SEQ ID NO: 13 - protein, 12 aa, Artificial Sequence
  R643 HCDR1
GFSLYRFDMGVG

SEQ ID NO: 14 - protein, 17 aa, Artificial Sequence
  R643 HCDR2
HIWWDDDKYYNPALQSR

SEQ ID NO: 15 - protein, 11 aa, Artificial Sequence
  R643 HCDR3
VRGLHDYYYYF

SEQ ID NO: 16 - protein, 10 aa, Artificial Sequence
  R643 LCDR1
CQASEDVSYM

SEQ ID NO: 17 - protein, 7 aa, Artificial Sequence
  R643 LCDR2
YGTSNKA

SEQ ID NO: 18 - protein, 8 aa, Artificial Sequence
  R643 LCDR3
CQQWSRRP

SEQ ID NO: 19 - protein, 123 aa, Artificial Sequence
  R783 heavy chain variable domain (VH)
QITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSS

SEQ ID NO: 20 - protein, 107 aa, Artificial Sequence
  R783 light chain variable domain (VL)
EIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKR

SEQ ID NO: 21 - protein, 123 aa, Artificial Sequence
  R725-2 heavy chain variable domain (VH)
QITLQESGPTLVKPTETLTLTCAFSGFSLYRFDMGVGWIRQPPGQGLEWLAHIWWDDDKYYNPALQSRLTISKDTSKNQVVLTMTNMDAEDTATYYCARVRGLHDYYYYFAYWGQGTLVTVSS

SEQ ID NO: 22 - protein, 107 aa, Artificial Sequence; identical to SEQ ID NO: 20
  R725-2 VL

SEQ ID NO: 23 - protein, 123 aa, Artificial Sequence; identical to SEQ ID NO: 21
  R643 heavy chain variable domain (VH)

SEQ ID NO: 24 - protein, 107 aa, Artificial Sequence
  R643 light chain variable domain (VL)
EIVLTQSPSTLSLSPGESVTLSCQASEDVSYMHWYQQKPGQAPQPWIYGTSNKASGVPSRFSGSGSGTDFTLTISSLEPEDVATYYCQQWSRRPFTFGQGTKVEIER